(19) United States
(12) Patent Application Publication
(10) Pub. No.: US 2018/0333056 A1
(43) Pub. Date: Nov. 22, 2018

(54) APPARATUS FOR MONITORING CARDIOVASCULAR HEALTH

(71) Applicant: Chang-An Chou, Taipei (TW)

(72) Inventor: Chang-An Chou, Taipei (TW)

(21) Appl. No.: 15/546,013

(22) PCT Filed: Jan. 25, 2016
(86) PCT No.: PCT/CN2016/071991

§ 371 Date: Jul. 25, 2017

(30) Foreign Application Priority Data
Jan. 26, 2015 (CN) 201510037439.6
Jan. 26, 2015 (CN) 201510037858.X
Jan. 26, 2015 (CN) 201510038040.X

(51) Int. Cl. A61B 5/0205; A61B 5/0408; A61B 5/00; A61B 5/04; A61B 5/0464; A61B 5/046

(52) U.S. Cl. CPC A61B 5/0205; A61B 5/04085; A61B 5/6815; A61B 5/6826; A61B 5/0006; A61B 5/4035; A61B 5/02405; A61B 5/7415; A61B 5/743; A61B 5/7455; A61B 5/0464; A61B 5/046; A61B 5/04012

(57) ABSTRACT

An apparatus and a method for monitoring cardiovascular health are provided. The apparatus includes a housing, a control circuitry, an inflatable cuff for surrounding an upper limb of a user, a pump contained in the housing, at least a first electrode and a second electrode, and an ear-worn structure having the first electrode mounted thereon. When performing blood pressure measurement, the processor controls the pump to inflate and deflate the cuff, for measuring blood pressure of the user. When performing electrocardiographic signal measurement, through mounting the ear-worn structure on an ear of the user, the first electrode contacts the skin of the ear or around the ear, and through mounting the cuff surrounding the upper limb, the second electrode contacts the skin of the upper limb, enabling the processor to acquire electrocardiographic signals through the first electrode and the second electrode.

### FIELD OF THE INVENTION

The present invention relates to an apparatus and a method for monitoring cardiovascular health, and more particularly to an apparatus for monitoring cardiovascular health capable of measuring blood pressures and electrocardiographic signals, and a method for monitoring cardiovascular health through the apparatus.

### BACKGROUND OF THE INVENTION

As modern people have paid more and more attention to health, particularly cardiovascular health, sphygmomanometers have become one of the most popular apparatus that people use at home to monitor cardiovascular health every day. Sphygmomanometers are convenient to use and helpful to prevent hypertension that is actually a dangerous factor responsible for cardiopathy, diabetes and many other chronic diseases.

Electronic sphygmomanometers represent one popular model of sphygmomanometers for home use. The operation involves first fitting its cuff, pressing down its start button, and waiting for automatic measurement of blood pressure to finish. Such a simple operation flow allows users to conveniently and regularly record blood pressure values and trends, thereby effectively monitoring their own cardiovascular health.

In recent years, as a result of users' increasing demands and technical development, in addition to the traditional function of measuring blood pressure, some sphygmomanometers have been developed to provide more information about the cardiovascular system, for example, arrhythmia information which can be derived arterial pulses. U.S. Pat. No. 7,020,514 proposes a sphygmomanometer that provides information of atrial fibrillation (AF).

Generally, determination of arrhythmia relies on electrocardiogram, and electrocardiogram is currently the most useful information source that accurately reflects heart activities. FIG. 1 is an electrocardiogram with a normal waveform. Therein, the P wave represents the process of atrial depolarization, the QRS complex reflects rapid depolarization of left and right ventricles, T wave represents rapid repolarization of the ventricles, the PR interval is the time period started from the P wave's to the start of QRS complex, which reflects the time required for the heart's electric signal moving from the sinoatrial node to the ventricles through the atrioventricular node, and the ST section represents slow repolarization of the ventricles. By observing changes of the waveforms, it is possible to know what happens at which part of the heart, thereby identifying which part of the heart is responsible for a symptom at issue.

Premature beats, for example, are recognized as one common symptom related to arrhythmia, and divided into two types, namely premature atrial contractions (PAC) happening in atrium, and premature ventricular contractions (PVC) happening in ventricle. A typical approach to distinguish these two apart is to observe whether the P wave and/or the QRS wave has any abnormal shapes, and thereby determine whether the systole is from the atrium or the ventricle. Since ventricular systole is responsible for pumping out the blood from the heart to all parts of the body, once there is abnormal ventricular systole, blood can not be pumped out normally, so as to cause abnormal blood supply. Thus, as compared to abnormal atrium systole, abnormal ventricle systole is a more serious symptom.

However, information about arrhythmia provided by a sphygmomanometer is based on heart rates obtained from arterial pulses. Usually, when there are abnormal heart rates observed, it is difficult to distinguish whether the abnormality is from atrium or ventricle by merely studying the waveforms of the arterial pulses. Consequently, the user is unable to know how serious the observed symptom is. Also, since arterial pulses measured at limbs are actually heartbeats transmitted to limbs through blood in blood vessels, the accuracy thereof is incomparable to an electrocardiogram. Thus, even if a sphygmomanometer indeed can conveniently screen some arrhythmic symptoms, unavoidably, the final determination of arrhythmia can only be done by observing an electrocardiogram.

Additionally, during blood pressure measurement, when inflation and deflation, the cuff applies different levels of pressures to blood vessels in the arm. If the pressure is too high, the amplitude of arterial pulses might be decreased due to the pressed blood vessels, and pulse measurement performed at this time may have omission that leads to erroneous determination. It is thus clear that when we use a sphygmomanometer's cuff to measure arterial pulses, there are many operational limitations, wherein it has to be consider the impact of the pressure variation from the cuff on the blood vessels, and it also has to consider that, for determination of arrhythmia, whether the diagnosis based on this is reliable.

Accordingly, when it comes to cardiovascular health, blood pressure measurement and electrocardiogram measurement should be both considered.

It is thus believed that an apparatus capable of measuring blood pressure and electrocardiographic signals at the same time will bring about huge improvement in the field of cardiovascular health monitoring, especially screening and determination of arrhythmia, and will also be able to provide convenience for related clinic diagnosis.

Sphygmomanometers represent one of the most common and popular apparatuses for monitoring cardiovascular health in family. As compared to sphygmomanometers, electrocardiographic signal measurement apparatuses designed for home use are rare in the market. If electrocardiogram measurement could be incorporated in a home-use sphygmomanometer, average users who have been used to measuring blood pressure at home can take electrocardiographic signal measurement at home as a part of their daily healthcare routine, and become more aware of their own cardiovascular health. Also, the two interrelated physiological signals can be used in a more effective way.

The existing home-use electrocardiographic signal measurement device is usually a hand-held one that allows a user to hold it with his/her hand and perform measurement. It adopts reusable dry electrodes that can contact skin directly without using electrode gel, and this is particularly convenient for home use.

One of the most common operation ways is the user has his/her one hand holding the device to contact the electrode on the surface thereof, and contacts the other electrode to his/her the other hand or torso, as shown in FIG. 2 and FIG. 3, so as to obtain electrocardiograms.

While hand operation is quite convenient, it brings about a huge challenge to accuracy. When the measurement is taken by making the electrodes contact a user's both hands, as shown in FIG. 2, since the user's hands may shake and tremble during measurement, the operation stability is problematic, and the measured electrocardiograms may have baseline shift and waveform deformation, as marked in FIG. 4A. Compared to the normal waveform of electrocardiographic signals, such baseline shift and waveform deformation can cause incorrect analytic results. Besides, when the user tries to stabilize his/her hands which causes muscle tension, or to make a special effort to ensure the contact between his/her hands and the electrodes, it will be very easy to generate electromyographic signals, as shown in FIG. 4B, thereby decreasing the signal quality and thus leading to incorrect electrocardiogram analytic results.

When the electrodes respectively contact the user's holding hand and the user's chest, as shown in FIG. 3, it will be more stable and can obtain stronger signals as compared to the foregoing two-hand operation method. However, its operation disadvantageously requires the user to remove his/her clothing over the chest, and this terribly limits where the measurement can be taken. Additionally, chest displacement due to respiration can also cause relative movement between the electrode contacting the chest and the electrode contacting the hand, and may similarly lead to baseline shift that makes the resulting electrocardiograms inaccurate.

Therefore, for incorporating electrocardiogram measurement to a sphygmomanometer, it is necessary to consider electrodes' type and configuration, so as to allow users to operate the equipment in an easy and convenient way, thereby facilitating to obtain good signal quality. The factors that affect signal quality mainly includes environmental interferences, instable contact between skin and electrodes, and the movement during measurement. For example, electromagnetic waves in the environment where the measurement is taken can cause noises in the obtained electrocardiographic signals, and electromyographic signals caused by instable contact or overtense muscles during measurement can also be artifacts. These all have adverse effects on signal quality. Further, as easy operation is another requirement, it is ideal to incorporate the motions of contacting electrodes into the normal operation flow of the existing sphygmomanometers, so as to prevent the trouble of relearning and to streamline operation process, thereby making users more willing to take measurement.

Hence, for making an ideal home-use apparatus for monitoring cardiovascular health, all the foregoing factors need to be considered when we incorporate electrocardiographic signal measurement into a sphygmomanometer.

The combination of blood pressure measurement and electrocardiographic signal measurement has another advantage that when stable and clear electrocardiographic signals are obtained, information about heart rate variability (HRV) is available, thereby allowing to know autonomic nervous system activities. Since the autonomic nervous system is also a factor that influences blood pressure, it wil be able to konw whether the cause of hypertension is related to autonomic nervous system by analyzing the relationship between the autonomic nervous system activities and blood pressure variation.

### SUMMARY OF THE INVENTION

One objective of the present invention is to provide an apparatus for monitoring cardiovascular health, which functions for both blood pressure measurement and electrocardiographic signal measurement.

Another objective of the present invention is to provide an apparatus for monitoring cardiovascular health, which introduces contact with electrodes required by electrocardiographic signal measurement to the established operation flow for blood pressure measurement, thereby minimizing operation complexity and maximizing user acceptance.

Another objective of the present invention is to provide an apparatus for monitoring cardiovascular health, which effectively and accurately provides information helpful to determine arrhythmia.

Another objective of the present invention is to provide an apparatus for monitoring cardiovascular health, which measures arterial pulses through cuff inflation for determining whether there is a possible arrhythmia event, and accordingly notifies user to perform electrocardiographic signal measurement, thereby providing the user electrocardiogram in real-time and thus facilitating further ensuring the occurrence and type of arrhythmia.

Still another objective of the present invention is to provide an apparatus for monitoring cardiovascular health, which adopts an ear-worn structure that actively applies a force to the user's skin at and around his/her ear, so as to ensure stable contact between the electrode thereon and skin.

A further objective of the present invention is to provide an apparatus for monitoring cardiovascular health, which adopts a finger-worn structure that actively applies a force to the user's finger skin, so as to ensure stable contact between the electrode thereon and skin.

Another objective of the present invention is to provide an apparatus for monitoring cardiovascular health, which combines electrodes with a cuff required for blood pressure measurement, so that when the cuff encompasses a user's arm, contact between the electrodes and skin is established simultaneously.

An additional objective of the present invention is to provide an apparatus for monitoring cardiovascular health, which has electrodes deposited on its housing's surface, so that when a user fits the cuff, contact between the electrodes and skin is established simultaneously.

Another objective of the present invention is to provide an apparatus for monitoring cardiovascular health, which has electrodes deposited on its housing's surface, so that when a user operates the apparatus, contact between the electrodes and skin is established simultaneously.

Yet another objective of the present invention is to provide an apparatus for monitoring cardiovascular health, which uses wearable structures to establish contacts between electrodes and skin, and thus, is suitable for long-term operation to acquire high quality electrocardiographic signals, thereby facilitating HRV analysis and identification of the relationship between autonomic nervous system activities and blood pressure.

### BRIEF DESCRIPTION OF THE DRAWINGS

FIG. 1 shows a standard waveform of electrocardiogram;

FIG. 2 illustrates one operation manner of a conventional hand-held electrocardiogram measuring apparatus;

FIG. 3 illustrates another operation manner of a conventional hand-held electrocardiogram measuring apparatus;

FIG. 4A shows electrocardiograms having baseline shift;

FIG. 4B shows electrocardiograms affected by electromyographic signals;

FIG. 5 is a block diagram of the apparatus for monitoring cardiovascular health according to the present invention;

FIGS. 6A-6C show exemplificative examples of ear-worn structure according to the present invention;

FIG. 7 shows skin around a user's ear to be contacted by the ear-worn structure according to the present invention;

FIGS. 8A-8B show exemplificative examples of finger-worn structure according to the present invention;

FIG. 9 is an exemplificative example showing the combination between an electrode and a cuff according to the present invention;

FIG. 10 is an exemplificative example showing the apparatus for monitoring cardiovascular health according to the present invention to have another housing for carrying a start button;

FIGS. 11A-11H are exemplificative examples showing the combination between electrode and housing according to the present invention;

FIGS. 12-15 show exemplificative examples of the apparatus for monitoring cardiovascular health according to the present invention, wherein electrodes are located on an ear-worn structure and combined with a cuff, respectively;

FIG. 16 shows an exemplificative example of the apparatus for monitoring cardiovascular health according to the present invention, wherein electrodes are located on an ear-worn structure and a wrist-worn structure, respectively;

FIGS. 17-19 show exemplificative examples of the apparatus for monitoring cardiovascular health according to the present invention, wherein electrodes are located on an ear-worn structure and a finger-worn structure, respectively;

FIG. 20 shows an exemplificative example of the apparatus for monitoring cardiovascular health according to the present invention, wherein electrodes are located on a finger-worn structure and combined with a cuff, respectively;

FIG. 21 shows an exemplificative example of the apparatus for monitoring cardiovascular health according to the present invention, wherein electrodes are located on a finger-worn and combined with the housing surface, respectively;

FIG. 22 shows an exemplificative example of the apparatus for monitoring cardiovascular health according to the present invention, wherein electrodes are located on two finger-worn structures, respectively;

FIGS. 23-24 show an exemplificative example of the apparatus for monitoring cardiovascular health according to the present invention, wherein electrodes are located on an ear-worn structure and combined with the housing surface, respectively;

FIGS. 25-27 show exemplificative examples of the apparatus for monitoring cardiovascular health according to the present invention, wherein electrodes are combined with the housing surface and with the cuff, respectively;

FIG. 28 shows an exemplificative example of the apparatus for monitoring cardiovascular health according to the present invention, wherein electrodes are located on an ear-worn structure and combined with the housing surface, respectively;

FIG. 29A shows an exemplificative example of the apparatus for monitoring cardiovascular health according to the present invention, wherein both electrodes are located on the housing surface;

FIGS. 30A-30B show exemplificative examples of the apparatus for monitoring cardiovascular health according to the present invention, wherein one electrode is located on the housing surface, and the other electrode is located on an ear-worn structure or on a finger-worn structure;

FIG. 31 is an operation flow chart of the apparatus for monitoring cardiovascular health according to the present invention; and

FIGS. 32-34 show exemplificative examples of notification of the apparatus for monitoring cardiovascular health according to the present invention.

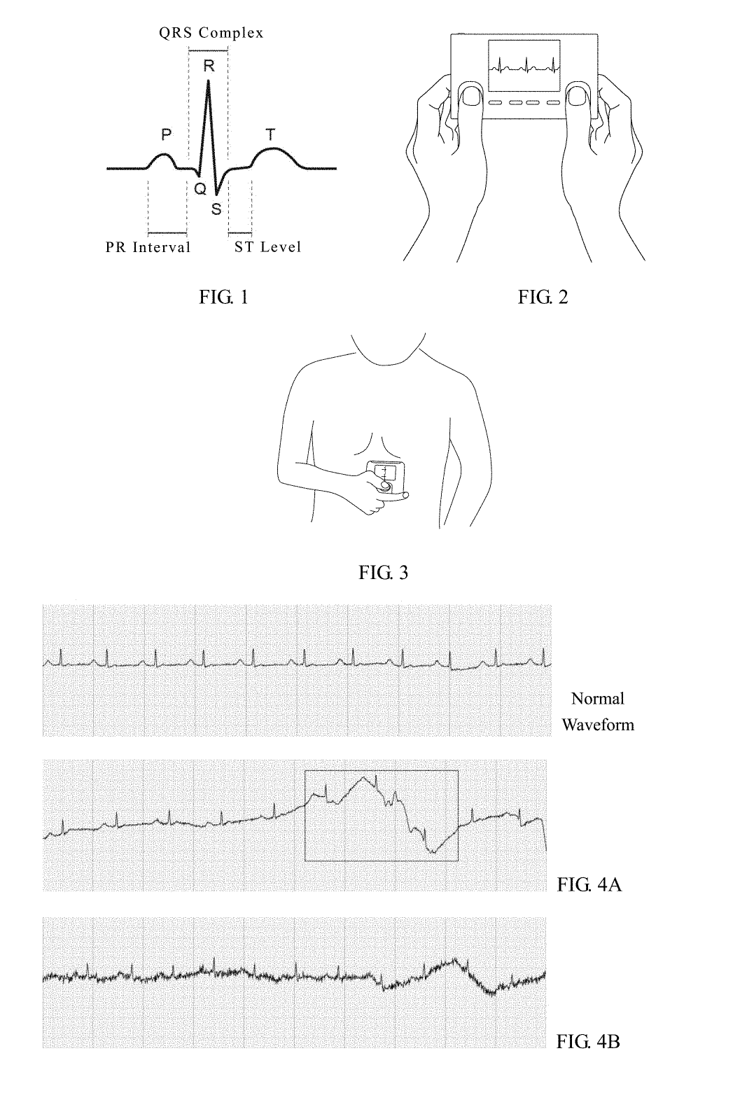

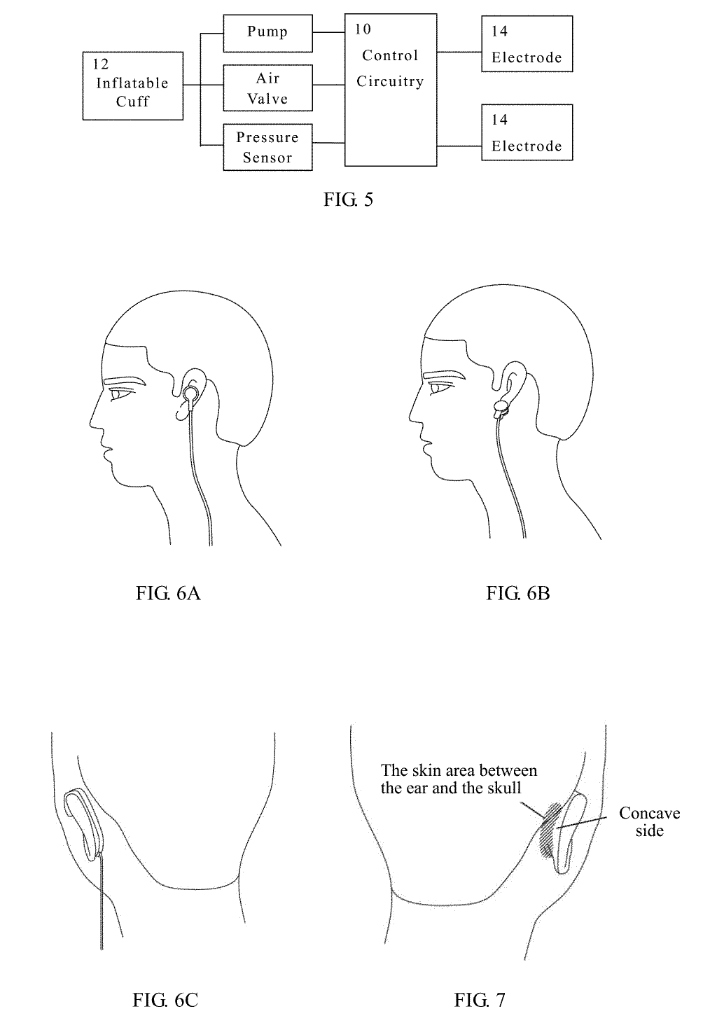

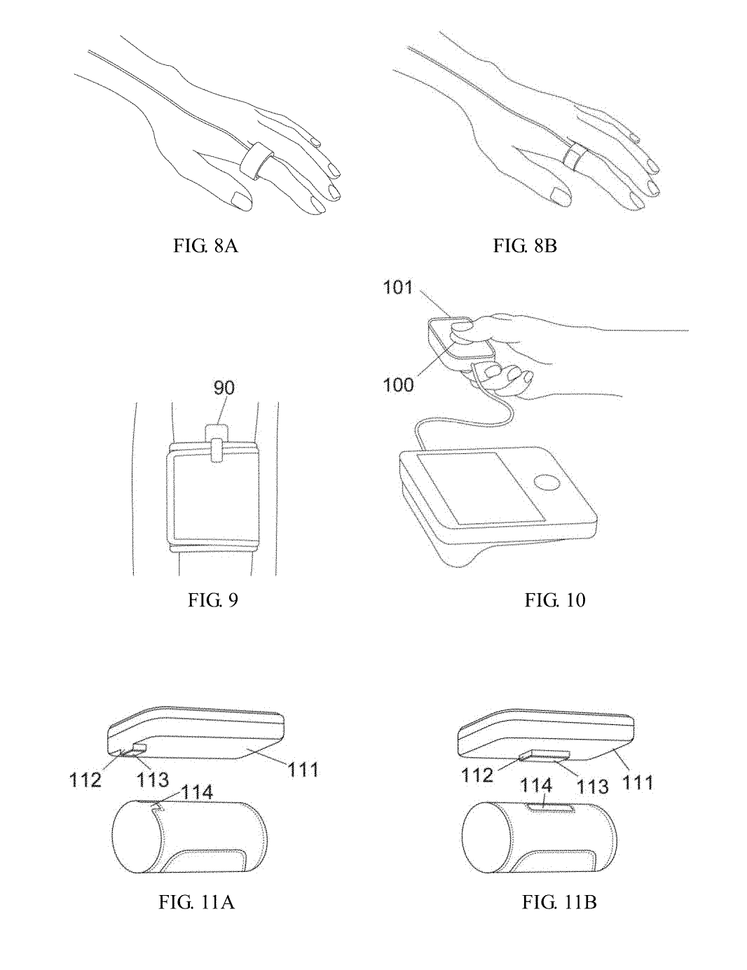

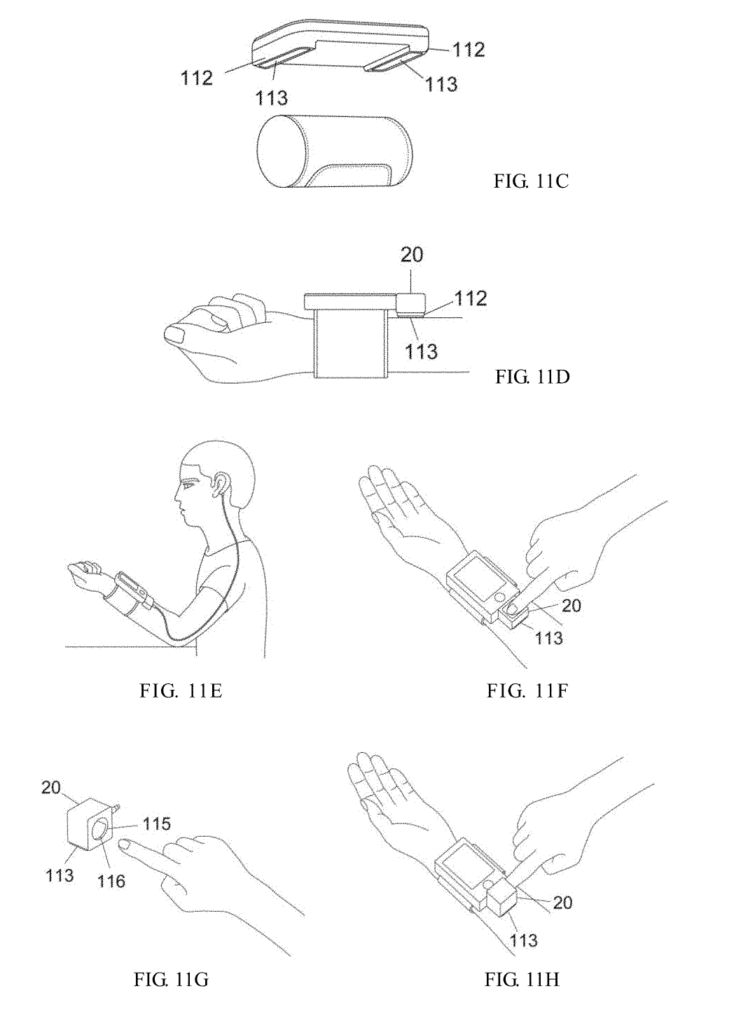

### DETAILED DESCRIPTION OF THE INVENTION

The present invention relates to an apparatus for monitoring cardiovascular health that functions for both blood pressure measurement and electrocardiographic (ECG) signal measurement. It allows a user to easily record electrocardiograms during the normal operation of blood pressure measurement he/she is used to. Thus, many kinds of important information about cardiovascular health can be obtained by operating a single apparatus.

Please refer to FIG. 5 first for a schematic view of an apparatus for monitoring cardiovascular health according to the present invention. As shown, the apparatus for monitoring cardiovascular health comprises a control circuitry 10, a cuff 12, a pump, an air valve, a pressure sensor, and at least two electrodes 14. Herein, the control circuitry 10 performs blood pressure measurement and electrocardiographic signal measurement using the cuff 12 and the electrodes 14 connected thereto. Therefore, the control circuitry 10 may also include, but not limited, some electronic components for achieving measurements, such as a processor, at least one A/D converter, a filter, an amplifier, and so on. As these are known to people of ordinary skill in the art, detailed description thereto is not discussed any further herein.

The disclosed apparatus for monitoring cardiovascular health also has a housing for containing the control circuitry and the pump. Herein, the housing may be combined with the cuff and thus be mounted on the user's body during measurement. Alternatively, it may be separated from the cuff and not mounted on the user's body during measurement. In addition, a user interface also can be implemented to located on the housing surface, such as a display element, a start button, input keys, and so on.

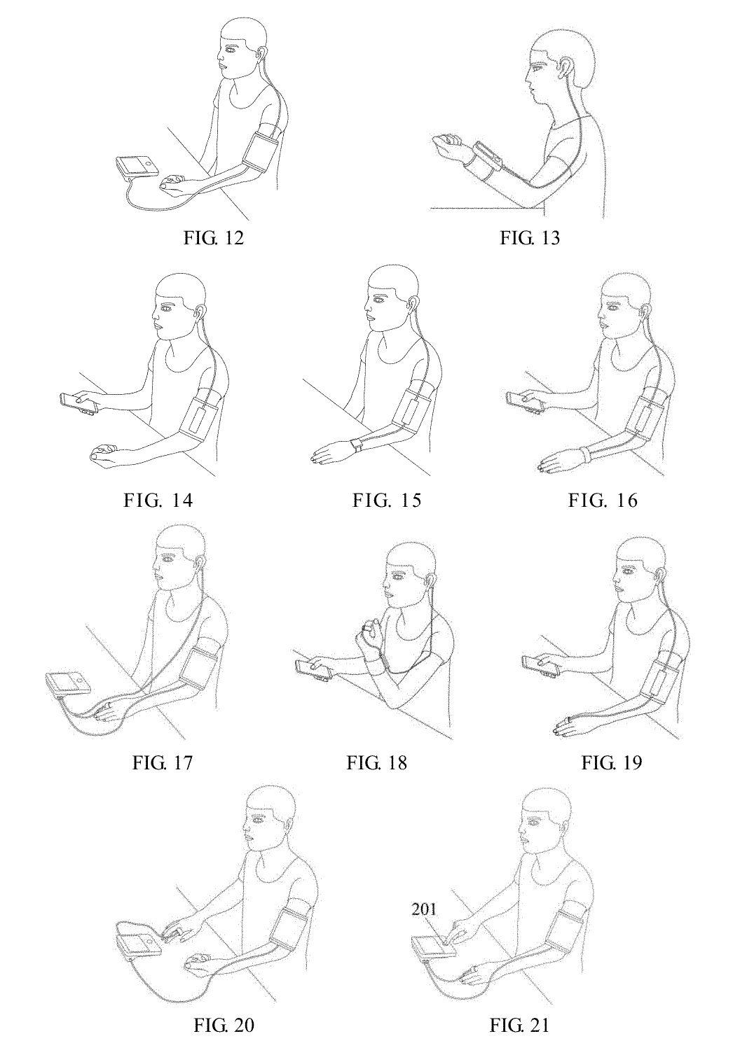

Since the disclosed apparatus for monitoring cardiovascular health provides measurement of electrocardiographic signals by adding electrocardiographic electrodes on the basis of blood pressure measurement, the present invention puts no limitation to its appearance and structure, and any existing electronic sphygmomanometer may be used as the basis for implementing the present invention. For example, an arm-type sphygmomanometer as shown in FIG. 12 and a wrist-type sphygmomanometer as shown in FIG. 13 are both suitable. In this way, users can perform electrocardiographic signal measurement according to the present invention during the operation he/she is used to.

In the present invention, measurement of electrocardiographic signals is achieved by using dry electrodes that contact human skin directly. The use of dry electrodes is more convenient, as compared to the traditional reusable wet electrodes, because user can perform electrocardiographic signal measurement without using and cleaning electrode gel. Thus, the measurement can be conveniently performed anytime. Additionally, as compared to disposable electrode patches, dry electrodes are more durable and easier to maintain, and since they are reusable, inconvenience and costs related to replacement of electrodes can be minimized. Dry electrodes used in the present invention may be, but are not limited to, electrodes made of stainless steel, conductive fabric, and conductive rubber, without limitation. Alternatively, the disclosed apparatus may work with electrodes that not directly contact human skin, such as, electrodes which acquire electrocardiographic signals capacitively, inductively, or electromagnetically, and similarly, this kind of electrodes also conveniently don't need to use electrode gel.

When integrating electrodes into a sphygmomanometer, the present invention takes the operation flow of blood pressure measurement as the basis, so that the user can feel familiar during operation, and it also mounts electrocardiographic electrodes on in a simple and ergonomic way, so as to ensure stable contact between the electrodes and the user's skin.

Typical operation of electronic sphygmomanometer involves: putting on the cuff on the user's arm or wrist, keeping the arm or wrist at the level equal to the heart, pressing the start button and staying still until the machine automatically finishes the measurement process.

In the foregoing flow, installing the cuff and pressing the start button are two indispensable steps. The concept of the present invention is to have the required motions for contacting electrocardiogram electrodes integrated into these necessary operation steps for blood pressure measurement, so as to prevent additional steps, and thus, the user does not need to learn a whole new operation flow.

Another consideration is where the electrodes are set on the sphygmomanometer. For ensuring good signal quality, the inventor selects the locations of the electrodes and the contact manner for the electrode and the skin with two considerations in mind. First, by properly selecting the contact location and designing the structure of electrode, the electrode can actively apply a force to contact the user's skin. In this way, contact between the electrode and the skin no more relies on the user's effort, and this not only improves contact stability, but also prevents from undesired electromyographic signals and artifacts. Second, where there is a need for the user's effort to contact the electrode, the present invention places the electrode at a proper and easy-to-contact position, so that the user can contact the electrode in a relaxed posture, thereby stabilizing contact and minimizing artifacts. This also helps to reduce muscle tension and prevent from undesired electromyographic signals. Additionally, if the electrodes are designed to have ergonomic contacting surfaces, contact stability can be further ensured, thereby significantly improving signal quality.

Accordingly, when considering where and how the electrodes are configured, the inventor has had the foregoing concepts in mind. One possible approach according to this basis is putting the electrode on the user's ear.

While an ear is not a body part usually recognized as one participating blood pressure measurement, there is a unique benefit of contacting the electrode with the ear because ear and its vicinity areas are where electromyographic signals are very weak. Besides, since the relative positional relationship between the ear and the head is very stable, even if the user moves his/her body during measurement, such as, slightly turning his/her body or neck, the contact between the electrode and the skin of ear or around the ear still can remain stable, without generating significant interferences that affect the measurement results.

Moreover, in our daily life, ears are less covered by clothing as compared to other body parts, and thus can be easily contacted, without the need of removing clothing thereon. In addition, there are fewer hairs on the ears or around the ears, thus allowing contact between the electrode and the skin to be established easily without hindrance. For these reasons, the ear is a convenient choice to most users.

Various fixing means may be implemented according to the ear's anatomy. For example, an ear plug, an ear clamp, and an ear hook, as shown in FIGS. 6A-6C, respectively, are all useful and common fixing means for attaching an article to an ear, and most users can easily put these on without relearning. In use, a user can install the electrode as easily as putting an earphone in the ear or clamping an earring on the earlobe. Through mounting the electrode on the ear by the means described above, contact between the electrode and the skin can be reliably established without user's force application, so the interferences caused from muscular tension and electromyographic signals can be minimized, thereby ensuring good signal quality.

The electrocardiographic signal can be obtained anywhere on the ear, without limitation. The measurement may be taken at any part of the ear, such as inside the canal, on the earlobe, at the concave side of auricle, e.g., the inferior concha, areas around the opening of canal, the helix, the convex side of auricle, and areas around the ear, as shown in FIG. 7, such as the skin area between the ear and the skull. All these locations are suitable sites for the electrode to contact and obtain the electrocardiographic signals.

Thus, the electrode may be extended from the machine or a structure combined with the cuff and configured to contact the ear. Therefore, after installing the cuff, the user can easily set the electrode in position to obtain good electrocardiographic signals.

Herein, the electrode may be put on either of a user's ears. However, according to experiments, where the other electrode is deposited is meaningful to signal quality. When the other electrode is deposited on the left upper limb, the obtained electrocardiographic signals have much greater quality than those obtained when the other electrode is on the right upper limb. Therefore, for electrocardiographic signal measurement with one electrode contacting the user's ear, the other electrode is preferably located to contact the left upper limb's skin, so as to prevent poor signal quality due to contacting the right upper limb and in turn erroneous analytic results.

In practice, the contact between the electrode and the ear may be established by an ear-worn structure configured to engage with the ear. Therein, the electrode is such deposited that it contacts the skin when the ear-worn structure is engaged with the ear. Thus, when the ear-worn structure is affixed to the ear, contact between the electrode and the skin of the ear or around the ear is established simultaneously.

The ear-worn structure may be realized in various forms. For example, when the ear-worn structure is in the form of an ear plug, the electrode may be deposited on the ear plug, so as to naturally contact skin in the canal, as shown in FIG. 6A. As an additional alternative, an ear plug with special design may be extended to match the profile inside the auricle. When the ear-worn structure is in the form of an ear clamp for clamping the auricle or the earlobe (FIG. 6B), the electrode may be deposited in the inner side of the ear clamp, so as to establish its contact with the auricle or the earlobe when the user wears the ear clamp. When the ear-worn structure is in the form of an ear hook, as shown in FIG. 6C, in one preferred embodiment, the electrode may be located on a hook extended to the back of the ear so as to contact the skin of the convex side of auricle or the skin area between the ear and the head. Herein, the hook may, for example, with its material's elasticity, or with a special structural design, apply a force toward the skin, thereby ensuring stable contact with the skin.

It is to be noted that, the aforementioned structural examples of the ear-worn structure are only illustrative and not limitations to the present invention. The present invention may combine any of these structures, for example, to combine two structures, e.g., an ear plug together with an ear hook. Its implementation may vary according to practical needs without limitation.

Alternatively, the ear-worn structure may be attached to the ear magnetically. For example, the ear-worn structure may have two components magnetically attracting each other with the ear standing therebetween, and the electrode may be deposited on the two components or one of the components. Herein, the two components may have magnetism. For example, they may contain a magnetic substance, or they may be a magnetic substance, or they may be made of a material that can be attracted by magnetism, or they may contain a substance that can be attracted by magnetism. For example, one of the components has magnetism, and the other component can be attracted by magnetism, or alternatively, the two components both have magnetism. There are actually various implementation possibilities, without limitation.

Additionally, in one preferred embodiment, the ear-worn structure and the electrode installed thereon may be connected to the cuff or the housing through a connecting port. In this way, when there is no need to perform electrocardiogram measurement, the ear-worn structure can be removed.

Herein, for preventing that the obtained electrocardiographic signals induct environmental noises through the connecting wire, the obtained signals may be processed upon its generation near the electrode by, for example, circuits for amplification, buffering, filtration, and/or digitalization, so as to ensure high signal resolution. Also, the necessary circuits may be further contained in the ear-worn structure, without limitation.

In addition, according to another aspect of the present invention, the electrode may also be carried by a finger-worn structure, such as a ring-like structure, or a band for encompassing the finger. The finger-worn structure is as advantageous as the ear-worn structure because wearing things on fingers is also familiar to most users and requires no additional learning. To perform measurement, the user only needs to directly put the finger-worn structure on his/her finger and the contact between the electrode and his/her skin can be established, so the operation flow is easy and convenient. In addition, since the contact between the electrode and the skin is achieved by the finger-worn structure applying a force to the finger, the user needs to relax his/her hand wearing the electrode and the possible interference from muscle tension can be minimized.

It is preferably that the finger-worn structure is combined with the finger at the knuckle where the phalanx proximalis or the phalanx media is, so as to prevent falling off from the hand due to being too close to the fingertip. In practice, the finger-worn structure may be in the form of a ring as shown in FIG. 8A, or in the form of a flexible band for encompassing the finger as shown in FIG. 8B, without limitation. Herein, no matter what the form used is, it may further have a structure for adjusting its encompassing diameter, so as to further ensure the stable contact between the electrode and the user's skin. For example, the ring may have a mechanism for adjusting its size so as to adapt to different wearers' fingers, and the band may have an adjustable fixing means, such as Velcro, for users to set how tight the encirclement is. These and the like may be implemented according to the actual situation without limitation. Alternatively, it may be a finger clamp that is designed to, for example, hold a finger at its tip or at other knuckles, like the phalanx proximalis or the phalanx media. In this case, fixation can be achieved by the springiness of the clamp, so it is also a useful scheme.

Herein, similar to the case of the ear-worn structure, when electrocardiographic signals are acquired at the finger, the signals may be processed as near as possible to the place they are obtained, so as to ensure signal quality. Similarly, the circuits may be installed inside the finger-worn structure.

Additionally, according to another aspect of the present invention, an alternative location to set the electrode is the cuff. Since placing the cuff around the arm or the wrist is necessary for taking blood pressure, when the electrode is located on the cuff, contact between the electrode and the skin can be established by installing the cuff, thereby simplifying operation. Herein, the electrode may be located at any part of the cuff, as long as its location allows contact between the electrode and the skin to be established when the cuff is placed around the arm or wrist. For example, the electrode may be located at the inner side of the cuff, or at the edge of the cuff, without limitation.

When the electrode is located at the inner side of the cuff, it may be an extensively used metal electrode. In one preferred embodiment, for improving its contact with the skin, the electrode also may be made of a flexible material, such as conductive fabric, conductive rubber, etc. Alternatively, it also may be a layer of conductive coating formed on the inner surface of the cuff, so that the electrode can bend with the cuff and contact the skin tightly.

For ensuring the contact between the electrode and the skin, it may be set that only when the inflation of the cuff reaches a pressure threshold (meaning that the contact force against the skin reaches a certain level), electrocardiographic signal measurement is performed, so as to further ensure the stability of the contact between the electrode and the skin.

Moreover, an additional structure may be provided to ensure the contact between the electrode and the skin by avoiding the influence of the cuffs inflation and deflation during blood pressure measurement. For example, a supporting structure may be installed on the cuff at a position corresponding to the electrode. Thus, when the cuff is placed around the arm or the wrist, its encompassing force or volume expansion caused by inflation can exert a force onto the supporting structure and in turn make the supporting structure apply a force toward the skin to the electrode, thereby ensuring the contact between the electrode and skin. For example, the supporting structure may have a certain thickness and hardness, so as to effectively transmit the cuff's encompassing force or expansion force to the electrode. Moreover, the supporting structure may have compressive elasticity, so that the applied force will not press strongly against the user's skin to cause uncomfortable feelings. In one preferred embodiment, the supporting structure is ergonomically designed to fit the skin it contacts for further ensuring contact stability. For example, the supporting structure may well fit the curve of the arm.

In another preferred embodiment, as shown in FIG. 9, the electrode 90 may be attached to the edge of the cuff, such as being clamped to the upper edge of the cuff. In this case, the contact between the electrode and the skin may be realized in various ways. For example, the electrode may be made of a material having elasticity so as to make the electrode apply a force toward the skin. Thereby, when the cuff is placed around the arm or the wrist, the electrode can naturally fit the skin. Alternatively, the electrode may be structurally designed to fit the profile of the arm or the wrist, so as to ensure the contact between the electrode and the skin. The actual implementation may vary according to practical needs without limitation.

It is to be noted that, when the electrode is combined with the cuff, electrocardiographic signal measurement and blood pressure measurement may be performed simultaneously. Alternatively, it also can be selected to perform electrocardiographic signal measurement and blood pressure measurement separately. Therefore, the user can choose according to the actual situation.

In addition, according to yet another aspect of the present invention, the other electrode is deposited on the surface of the housing for a user to contact with his/her finger.

For performing blood pressure measurement, after the cuff is set up well, it is necessary to press the start button on the housing for activating inflation and measurement. Therefore, when the electrode is combined with the start button, a user can easily press and hold the start button to establish contact with the electrode, thereby simplifying the operation steps of electrocardiogram measurement.

Furthermore, the finger's pressing of the start button may also be implemented to activate electrocardiographic signal measurement and blood pressure measurement simultaneously. In this way, a single press can realize three procedures simultaneously, namely contacting the electrocardiogram electrode, activating blood pressure measurement, and activating electrocardiographic signal measurement, thereby minimizing operational complexity.

Herein, the start button may be a button with a pressing stroke or a touch button, without limitation. The surface of the start button may be ergonomically shaped to fit the profile and curve of the finger, making the contact more stable.

In use, a user may choose to only perform blood pressure measurement or electrocardiographic signal measurement, or perform both simultaneously. For example, different operational options may be switched by different pressing strokes of the start button, or by changing the duration the button is pressed. For example, a brief press means there is no need to establish contact with the electrode, so only blood pressure measurement is activated, and a long press activates electrocardiographic signal measurement, while a brief press immediately followed by a long press activates the two types of measurement simultaneously. The operation may vary according to the actual implementation without limitation.

In a preferred embodiment, as shown in FIG. 10, the start button 100 may be carried by another housing 101 other than the housing, such as a press-driven structure. In this way, the start button may be relocated to different sites according to the user's operational preference, thereby allowing the user to perform electrode contact with a more relaxed posture, and this is also contributive to good signal quality.

Additionally, according to another aspect of the present invention, when the housing is carried by the cuff, the electrode may be located on various parts of the housing, provided that it can be implemented to locate at a position where contacts the skin when the cuff is placed around the limb.

When the housing is carried by the cuff and fits around the upper arm or the forearm (as shown in FIG. 13 and FIG. 14), a carrying structure 112 may be further included to mount on the housing. For example, it may be located on the surface 111, as shown in FIGS. 11A-11C, so as to contact skin of the user's upper arm or forearm when the cuff encompasses the limb. Thus, when the electrode 113 is deposited on the carrying structure 112, the electrode contact can be established when the cuff is placed.

For example, as shown in FIG. 11A, the carrying structure 112 may be located near the edge of the cuff, and the cuff may have an opening 114 at a position corresponding to the carrying structure. Thus, by placing the cuff around the upper arm or the forearm, the contact between the electrode 113 and the skin can be established simultaneously. Alternatively, as shown in FIG. 11B, the opening 114 may be located within the cuff, and the carrying structure 112 positionally corresponds thereto. In addition, as shown in FIG. 11C, the carrying structure 112 may be alternatively located at two lateral edges of the cuff, and in this way, the contact with the skin can be established without structurally modifying the cuff. While, in the drawing, there are two carrying structures at the two lateral edges, it is also possible to have only one carrying structure provided at one lateral edge.

Additionally, the carrying structure may be implemented to have elasticity so as to absorb possible displacement occurring during inflation, thereby ensuring stable contact between the electrode and the skin. For example, it may be made of an elastic material, such as rubber, silica gel, and so on. Alternatively, it may be provided with a retractable mechanism, such as a button structure with pressing stroke. There are various possibilities.

It is to be noted here that while the carrying structure may be a raised part as shown, it is not limited thereto, and may be shaped depending on how the housing and the cuff are combined. For example, the carrying structure may be flush with the surface of the housing, as long as the contact between the electrode and the skin can be established when the cuff is placed around the arm, without limitation.

Alternatively, as shown in FIG. 11D, the carrying structure 112 may be located on another housing 20 and be deposited on the housing through a mechanical combination between the other housing and the housing, thereby when the cuff is placed around the upper arm or the forearm allowing the electrode 113 to contact the skin.

Importantly, in addition to mechanical combination, an electrical connection between the other housing and the housing also will be achieved, so that the electrode 113 can work with the other electrode to perform electrocardiographic signal measurement. The electrical connection may be achieved by a pair of connectors located on the other housing and on the housing, respectively. For example, they may be USB connectors or mini USB connectors. In this case, the mechanical combination can directly achieved by the pair of electrical connectors. Alternatively, the mechanical combination may also be accomplished by using matched physical structures respectively on the other housing and on the housing, without limitation.

The other electrode may be of any of the foregoing forms, as long as the skin it contacts is located at a body portion other than the limb encompassed by the cuff. For example, it may be an ear-worn electrode, a finger-worn electrode, or an electrode located on the start button.

Particularly, except that the other electrode is connected to the housing or located on the housing, and it also can be connected to the another housing through a connecting wire, or directly located on the another housing, that is, the two electrodes for performing electrocardiographic signal measurement are both provided by the another housing. For example, in addition to the electrode 113 located on the carrying structure, the another housing may be further connected to an ear-worn electrode (as shown in FIG. 11E), or connected to a finger-worn electrode. Additionally, the other electrode may be deposited on any surface of the another housing other than the surface having the electrode 113 mounted thereon, as shown in FIG. 11F, so as to be contacted by the other hand to perform the electrocardiographic signal measurement. Furthermore, the other electrode may be located on the foregoing start button for convenient operation.

In addition, as shown in FIGS. 11G-11H, the other housing 20 may be provided with an indentation 115, such as an annular indentation or a depression for a finger to put in and contact the other electrode 116 deposited on its inner surface. The inner surface is implemented to have a surface contoured to the finger, so that when putting in the indentation, the finger skin can contact the electrode. Herein, the indentation may be made of an elastic material, such as rubber or silica gel, so as to achieve the contact between the electrode and the skin. Alternatively, it may be formed to have a plastic housing lined with an elastic material for encircling the finger or having a structure that provide an inward force, so as to ensure good contact between the inner electrode and the fingertip skin, without limitation.

Preferably, at least some circuits for acquiring electrocardiographic signals may be contained in the other housing, such as circuits for amplification, buffering, filtration, and/or digitalization. And, since the another housing and the housing are detachably combined through mechanical combination, when the two electrocardiogram electrodes are both deposited on the another housing, simply by attaching the another housing, the user can add his/her blood pressure measuring apparatus with the function of electrocardiogram measurement, which is extremely convenient.

In addition to the locations and methods described above for setting the electrodes, the disclosed apparatus for monitoring cardiovascular health may also use electrodes of any other forms contributive to minimized electromyographic signals and maximized contact stability. For example, the electrode may be carried by a wrist-worn structure and configured to contact the wrist. This configuration ensures contact between the electrode and the wrist's skin without using the user's exerting force, and is thus very ideal. The user just has to relax his/her encompassed limb during measurement, and good quality signals can be obtained.

The foregoing configurations are only exemplificative and can be implemented on any of the electrocardiographic electrodes according to practical needs without limitation. Some embodiments will be described below for further explaining the present invention.

Please refer to FIG. 12 and FIG. 13. According to one embodiment of the present invention, the two electrodes for electrocardiographic signal measurement are deposited on the inner side of the cuff and on an ear-worn structure, respectively. Thus, for blood pressure measurement, after setting up the cuff, the user can easily finish his/her preparation for blood pressure measurement and electrocardiogram measurement by wearing the ear-worn structure. This is almost the same to the case of the conventional approach to measuring blood pressure, with the only difference relying on the addition of a motion for putting on the ear-worn structure like putting on a conventional earphone. Thus, a user can finish the operation easily and effortlessly. Herein, the ear-worn structure may be connected to the cuff or the housing, without limitation.

Additionally, FIG. 14 and FIG. 15 illustrate how to use external apparatuses as the information display interface. For example, a smart phone, a tablet computer, or a smart watch may be used to externally display information. In this way, the housing carried by the cuff can be downsized, thereby providing the user more comfortable using experience. Herein, the connection between the housing on the cuff and the external apparatus may be wired or wireless connection, such as USB, Bluetooth, or Wi-Fi connection, without limitation.

In FIG. 14, the external apparatus is a smart phone for wireless connection, and in FIG. 15, the external apparatus is a smart watch for wired connection. Herein, the external apparatus may have more functions besides receiving data and displaying in a real-time, such as, guiding the operation and displaying the measurement results. In one instance, the external apparatus is capable of controlling operation of the disclosed apparatus, activating blood pressure and/or electrocardiogram measurement, analyzing the received data, storing the data and outputting the data to another apparatus, so as to provide more convenience. Such a configuration is particularly advantageous when the housing is carried by the cuff, because the user can easily activate measurement, learn the operation flow, and view the measurement results through the external apparatus, thus being very convenient.

FIG. 16 shows an example wherein the electrodes are mounted on an ear-worn structure and on a wrist-worn structure, respectively. As shown, the wrist-worn structure may be formed as a bracelet. Alternatively, it may be in the form of a band. Alternatively, the electrode may also be located on the inner side of the watchstrap of the smart watch as shown in FIG. 15, without limitation. In this case, a user just needs to wear the ear-worn structure and the wrist-worn structure and establish the contacts of the electrodes with the skins, measurement of electrocardiographic signals can be performed. This scheme similarly provides easy operation and good signal quality.

In the aforementioned embodiment, advantageously, during the entire process of electrocardiogram measurement, the contact between the electrodes and the skin is achieved and maintained without the user's exerting force actively, so as to prevent interference caused by electromyographic signals, thereby being beneficial to acquire signals with good quality. It is to be noted herein that, in such a configuration, the ear-worn electrode may be selectively worn on either the left or right ear, without limitation. However, as described previously, the location of the other electrode has effects on the signal quality to some extent, so the cuff should encompass the left upper limb for better signal strength.

According to another embodiment of the present invention, as shown in FIGS. 17-19, the two electrodes may be installed on an ear-worn structure and on a finger-worn structure, respectively. A user who wants to perform electrocardiographic signal measurement just wears the structures on his/her ear and finger, respectively, the contact between the electrode and the skin can be easily established. The contact between the ear-worn structure or the finger-worn structure and the skin does not involve the user's exerting force, so interference from electromyographic signals can be minimized. Additionally, since the wearing motion is very convenient, and there is no need for using a cuff, this scheme is very suitable for pure electrocardiographic signal measurement.

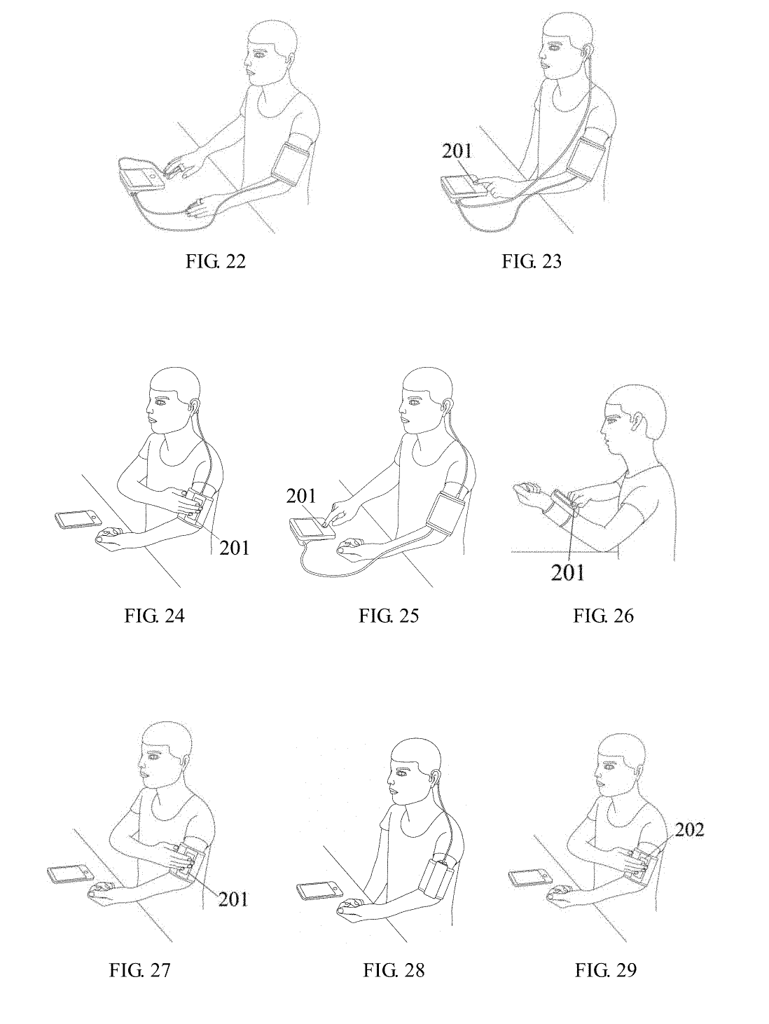

Additionally, in the case where one electrode is installed on the finger-worn structure, the electrode may work with another electrode installed at a different site to perform electrocardiographic signal measurement, such as an electrode inside the cuff (FIG. 20), or an electrode 201 on the housing surface (FIG. 21). Thus, a user only needs to add a motion of wearing the finger-worn structure on his/her finger to the normal operation flow for blood pressure measurement, so the operation is very convenient. Additionally, the two electrodes may also be carried by two finger-worn structures, as shown in FIG. 22. This is also a very convenient way. Since there is no need of using a cuff, this scheme similarly suits for pure electrocardiographic signal measurement.

According to another embodiment of the present invention, as shown in FIG. 23, the two electrodes for measuring electrocardiographic signals include one electrode 201 located on the surface of the housing having user interface and combined with the start button, and the other electrode combined with an ear-worn structure. With this configuration, a user, after setting up the cuff, can easily obtain blood pressure reading and the electrocardiogram by wearing the ear-worn structure, pressing the start button, and maintaining the contact between the finger and the start button.

In addition, as shown in FIG. 24, even if the data is to be transmitted to an external apparatus through wireless connection, an electrode 201 still can be provided on the housing, which is located at the upper arm, and combined with the start button, so as to achieve electrode contact and activate electrocardiogram measurement by pressing. Alternatively, measurement may be activated through an external apparatus, such as a smart phone, and the electrode located on the housing surface is merely for performing electrocardiogram measurement. The present invention puts no limitation thereto.

According to still another embodiment of the present invention, the two electrodes for electrocardiographic signal measurement are one electrode combined with the start button and the other electrode combined with the cuff, as shown in FIG. 25. A user can sit by a desk, like using a normal arm-type sphygmomanometer, with his/her left upper arm encompassed by the cuff and laid on the desk relaxedly, and use his/her right hand to press the start button 201 of the sphygmomanometer, thereby activating blood pressure measurement. And, with the special design of the present invention, the contacts of at least two electrodes (i.e. the electrode in the cuff and the electrode located on the start button on the housing surface) required by electrocardiogram measurement with skin of different body portions are at the same completed in this normal blood pressure measuring operation, without any additional steps. In other words, a single operation can obtain two kinds of physiological signals at the same time. Alternatively, as shown in FIG. 26, when the sphygmomanometer used is a wrist-type sphygmomanometer, the housing carried by the cuff is located at the wrist. In this case, a user may similarly contact the electrode located on the housing surface by simply pressing the start button 201, for cooperating with the electrode inside the cuff to perform measurement, thereby obtaining two kinds of physiological signals in a single operation. Alternatively, as shown in FIG. 27, in the case where data is to be transmitted to an external apparatus through wireless connection, it also can employ an electrode 201 combined with a start button on the housing carried by the cuff to cooperate with the other electrode installed on the inner side of the cuff, so as to achieve contact and activate electrocardiogram measurement by a simple pressing.

In this case, as compared to the process of only performing blood pressure measurement, the user just has to make the finger and the start button contact longer when he/she wants to measure electrocardiographic signals. As there is no need for additional steps, the operation is easy and effortless.

It is to be noted here that while contact with the start button comes with contact with the electrode, the user can still choose to perform blood pressure measurement or electrocardiographic signal measurement separately. For example, the user may choose the measurement he/she wants by varying the contact time, without limitation.

Instead of being located on the start button as described previously, the electrode may be alternatively located on the other portion of the housing surface. As shown in FIG. 28, the housing has the structure as shown in FIG. 11C, and the electrode is mounted on the carrying structure, which is located on the surface where the housing is combined with the cuff. Consequently, the step of setting up cuff can establish contact between the electrode on the housing and the upper arm's skin, and with wearing the wear-worn structure, the user can establish the contacts of both electrodes in an effortless manner without exerting force. As compared to the normal operation flow for blood pressure measurement, there is only one step of wearing the ear-worn structure added, thus being very convenient.

Additionally, the two electrodes may be located on the same housing, as shown in FIG. 29. Therein, the housing has a structure as that shown in FIG. 11B. Thus, when the cuff surrounds the upper arm, the electrode facing the upper arm can naturally pass through the cuff and contact the skin of the upper arm, while the other electrode 202 located on the housing surface can contact the other hand, so as to enable measurement of electrocardiographic signals. It is to be noted herein that while the electrode 202 as shown is located opposite to the surface facing the upper arm, it may be alternatively located on any surfaces other than the surface facing the upper arm, as long as its location is favorable to its contact with the user's the other hand. For example, it may be located on a surface adjacent to the surface facing the upper arm, without limitation.

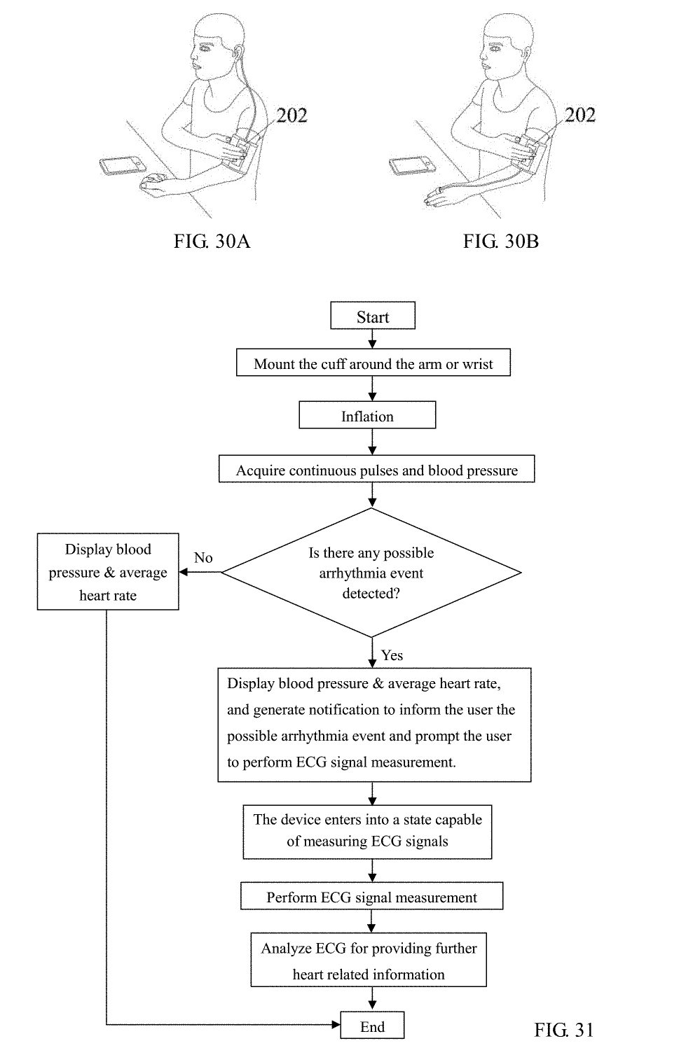

Furthermore, it is possible to allow a user to choose the electrodes he/she prefers. For example, the electrode facing the upper arm may be replaced by a switch (not shown) or connected to the other electrode, such as an ear-worn structure (as shown in FIG. 30A) that has an electrode, or a finger-worn structure (as shown in FIG. 30B) that has an electrode. In this way, a user can choose the most suitable use according to individual needs, making the use more convenient.

Moreover, there may be more than two electrodes used. For example, a third electrode acts as the ground or a reference electrode for suppressing common-mode noises, such as noises from power source, and may be implemented using any of the aforementioned electrode designs.

Additionally, in the present invention, for facilitating electrocardiogram measurement, the electrode may be partially or entirely connected to a sensor, for detecting and informing the user of whether the contact between the user and the electrode is appropriate. For example, a pressure sensor is used to detect the size of the force applied to the electrode, or impedance check is performed to determine whether the electrode is in contact and whether the contact is good. A simple alternative is to use a switch to sense the force applied to the electrode. In this case, when the control circuitry determines that the contact on the electrode satisfies a predetermined criterion, such as, a force large enough, and/or the electrode has been contacted, and/or the contact is good, it will allow electrocardiogram measurement to start automatically, or even allow the apparatus to be activated.

On the other hand, for providing users with an operation flow smoother and more convenient, a sensor may be arranged near an electrode to detect whether the electrode has been placed at a predetermined location, such as whether the ear-worn structure has been on the ear, whether the finger-worn structure has been on the finger, whether the electrode on the carrying structure has been put on the arm, and whether the cuff has encompassed the arm. Herein, the sensor may be implemented to be capacitive, resistive, or light sensing typed sensor, without limitation. Additionally, sound or screen display may be further implemented to inform the user that the electrode has been placed at the predetermined location, thereby making the operation even easier.

With this design, the sensing or detection for checking whether electrode contact is good may be performed after it is confirmed that the electrode has been placed at the predetermined location. Similarly, sound or screen display may be implemented to inform the user of the establishment of electrode contact, making the overall operation flow even smoother.

Thus, with the positions of electrode combined with a sphygmomanometer as proposed by the present invention, a user can achieve the arrangement of electrodes required for electrocardiographic signal measurement while using the sphygmomanometer in an easy and convenient way, thereby recording electrocardiograms effortlessly. Since electrocardiograms can provide detailed electrical activities about the heart, the disclosed apparatus thus can provide more detailed and more accurate information about cardiovascular health. For example, by performing an algorithm preloaded in a processor in the control circuitry, or by transmitting the electrocardiogram to an external apparatus and then performing an algorithm loaded therein, it is possible to determine the type of arrhythmia, such as PAC or PVC, and other arrhythmia-related symptoms can also be identified, such as atrial fibrillation (AF), bradycardia, tachycardia, and pause. Other symptoms in addition to arrhythmia may be also detected. For example, the ST level indicates existence of myocardial infarction, and the amplitude of QRS wave reveals if ventricle hypertrophy is existed.

Furthermore, with the relevance between blood pressure readings and electrocardiograms, cross reference between the two kinds of signals is useful to get information about other physiological conditions, such as PTT (Pulse Transit Time, which is the time a pulse wave propagates through a length of an artery). Additionally, the comparison between arterial pulses and electrocardiographic signals is helpful to remove noises/artifacts, so as to obtain correct interpretation of various cardiovascular information.

Additionally, the disclosed apparatus for monitoring cardiovascular health may also provide information on heart rate variability (HRV) according to the obtained electrocardiographic signals, so as to let the user understand ANS activities. The reason is that, the autonomic nervous system is one of the factors that influence blood pressure. When sympathetic nervous system is more active, blood vessel systole makes blood pressure increase. On the contrary, when parasympathetic nervous system is more active, blood pressure decreases.

With the function of electrocardiographic signal measurement, the disclosed apparatus can obtain accurate RRI (R-R Interval) sequences, namely heart rate variation, and obtain HRV through calculation, so as to perform HRV analysis and provide information about autonomic nervous system activities. Plus blood pressure measurement, a user can learn the relationship between blood pressure and autonomic nervous system in a real-time manner. For example, the user may determine whether his/her hypertension is related to autonomic nervous system, and, if the relationship is confirmed, the user may learn whether his/her physiological and psychological adjustment, such as relaxation and breathing training, is positively affecting his/her autonomic nervous system, thereby improving blood pressure.

Which kind of HRV analysis to be performed may be subject to practical needs. For example, frequency domain analysis may be performed to obtain the total power (TP) that is useful to evaluate the overall heart rate variability, the high frequency power (HF) that reflects parasympathetic nervous system activity, low frequency power (LF) that reflects sympathetic nerve activity or modulation results of sympathetic nerves and parasympathetic nerves, and LF/HF (ration of low and high frequency power) that reflects activity of sympathetic/parasympathetic nervous system. Additionally, after the frequency analysis, by checking the frequency distribution, the user may know the harmony of function of autonomic nervous system. Time domain analysis may also be performed to obtain SDNN that indicates the overall heart rate variability, SDANN that indicates the long-term overall heart rate variability, RMSSD that indicates the short-term overall heart rate variability, and R-MSSD, NN50 and PNN50 that may be used to evaluate high frequency variability in heart rate variability.

It is to be noted herein that, the procedure for obtaining RRI sequence through electrocardiographic signals may be performed before or after blood pressure measurement, as long as it can reflect the current relationship between blood pressure values and autonomic nervous system in a real-time manner, without limitation. Additionally, since HRV analysis requires relative long sampling time, typically 5 minutes, and the user has to be in a relaxed state, this is preferably performed in the case where the contact between the electrode and the skin is established and maintained without the user's exerting force. For example, it may be performed using the disclosed apparatus having the electrodes mounted on an ear-worn structure or a finger-worn structure, or having the electrode that contacts the user's skin when the cuff is placed around the user's arm or wrist. The implementation may vary according to the user's preference, without limitation.

Upon completion of measurement, the disclosed apparatus for monitoring cardiovascular health may inform its user of the measurement results, such as blood pressure readings, the average heart rate, arrhythmia indication, and heart rate variability, through a display element. Additionally, the disclosed apparatus may also comprise a memory for storing signals, analytic results, and/or relevant information. In one preferred embodiment, the memory is a removable memory, so that the user can transmit data conveniently or take the removable memory with stored measurement/analytic results to see the doctor. Additionally, the disclosed apparatus may further comprise a communication module for performing wired communication, such as USB connection, or wireless communication, such as Bluetooth or Wi-Fi, so as to transmit the obtained signals, measurement/analytic results and other data to an external apparatus, such as a personal computer, a smart phone, a tablet computer, a smart watch and so on, for display and/or further calculation and analysis. Herein, the transmission with the external apparatus may also be implemented to be real-time transmission, without limitation.

With the arrangement of the electrodes as proposed by the present invention, a user can use record electrocardiograms effortlessly and conveniently during blood pressure measurement. However, arrhythmia does not happen every time performing the blood pressure measurement, but blood pressure values are physiological signals that need to be recorded regularly and consistently. Thus, according to another aspect, the present invention further provides a mechanism to pre-screen whether there is any arrhythmia event under the situation that only blood pressure measurement is performed. In this way, a user may choose to perform electrocardiographic measurement only when the screening result suggests a possible arrhythmia event.

The basis of the pre-screening is that during measurement of blood pressure, the cuff's inflation can allow not only to measure blood pressure values but also to detect arterial pulses. Thus, by analyzing consecutive arterial pulses, it will be able to obtain heart beats corresponding to the pulses, so as to further identify whether there is any possible arrhythmia event, such as premature beats, artrial fibrillation (AF), tachycardia, bradycardia, pause, etc.

To achieve the foregoing objective, the disclosed apparatus for monitoring cardiovascular health further includes an arrhythmia detecting unit, a notification generating unit, and an electrocardiogram analyzing unit.

The arrhythmia detecting unit is capable of determining whether there is a possible arrhythmia event according to consecutive arterial pulses obtained through the cuff during blood pressure measurement. The notification generating unit is capable of generating notification during and/or after blood pressure measurement to inform the user of any possible arrhythmia event, and prompting the user to perform electrocardiographic signal measurement. The electrocardiogram analyzing unit provides more heart-related information by analyzing the obtained electrocardiogram. For example, by analyzing the waveform, it is possible to know information like the type of arrhythmia and whether there are other heart symptoms.

As shown in FIG. 31, when a user performs blood pressure measurement, he/she may put the inflatable cuff around his/her limb, e.g., the upper arm or the wrist, as what is done for normal blood pressure, and then start inflation. At this time, in addition to blood pressure readings, arterial pulses can be obtained simultaneously. Thus, the arrhythmia detecting unit can use the obtained pulses to determine whether there is a possible arrhythmia event. If there is no possible arrhythmia events detected, the user is informed of the measured blood pressure values and the average heart rate as that is seen in a normal blood pressure measurement process. If it is determined that there is a possible arrhythmia event, in addition to the information available form blood pressure measurement, such as blood pressure readings and the average heart rate, the notification generating unit generates notification to inform the user of the possible arrhythmia event in a real-time manner and to prompt the user to perform electrocardiographic signal measurement. At the same time, the disclosed apparatus for monitoring cardiovascular health enters into a state capable of measuring electrocardiographic signals, so as to allow the user to record the current electrocardiogram. Then, the electrocardiogram analyzing unit analyzes the electrocardiogram to further provide the user with more information of heart conditions.

In this way, the user can perform blood pressure measurement without changing his/her habit of measuring blood pressure, and only needs to perform electrocardiographic signal measurement and record an electrocardiogram, by contacting the electrodes integrated with the sphygmomanometer, when there is a possible arrhythmia event, so as to immediately know the analytic results of the electrocardiogram. Thus, the present invention features not only convenient measurement, but also the capability of providing more accurate arrhythmia-related information.

It is to be noted herein that, since the determination of a possible arrhythmia event is done by analyzing arterial pulses, it is possible to obtain arterial pulses using the cuff's inflation without measuring blood pressure, which has identical effect. Thus, the process may vary according to the user's practical needs, without limitation.

It is also to be noted that, when the cuff is inflated to obtain arterial pulses during blood pressure measurement, under-inflation may disable the apparatus from taking the pulses and over-inflation may press the blood vessels excessively and decrease measurement accuracy, so that, in practice, detection of arterial pulses may be set to perform only when a certain criterion of cuff inflation is satisfied. For example, a program may be used to control that the detection is performed under constant inflation pressure. Alternatively, it may be such set that the pulse measurement is only performed when the inflation has reached a certain pressure value (or the contact force is greater than a certain level).

After consecutive arterial pulses are obtained, the arrhythmia detecting unit analyzes the consecutive arterial pulses by first calculating time intervals between each two consecutive pulses so as to obtain a time-sequence characteristic of pulses, comparing the time-sequence characteristic to the known time-sequence characteristics of various arrhythmia types, such as premature beats, AF, bradycardia, tachycardia, and pause, and determining existence of a possible arrhythmia event when the characteristics match.

Advantageously, in the present invention, when detection of the possible arrhythmia event, the sensitivity can be properly increased by adjusting parameters of algorithm. Since the possible arrhythmia event can be instantly confirmed by analyzing the electrocardiogram obtained in the subsequently performed electrocardiographic signal measurement, even if the sensitivity is high, it is unlikely to have erroneous determination. Thus, the present invention can easily achieve highly accurate determination and effectively eliminates erroneous determination as seen in the prior art.

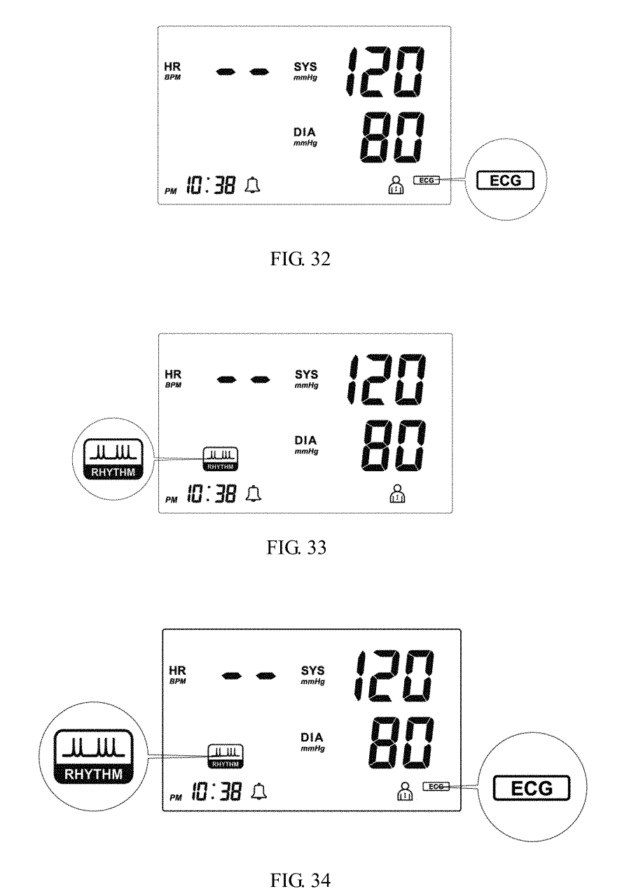

When the arrhythmia detecting unit determines that there is a possible arrhythmia event, the notification generating unit generates notification to inform the user of the possible arrhythmia event and to prompt the user to perform electrocardiographic signal measurement. The notification may be generated during and/or after pulse measurement, without limitation. The contents and ways of notification may also vary depending on real demands. For example, in one preferred embodiment, a symbol of ECG measurement in the screen may be lit after blood pressure measurement, as shown in FIG. 32, so as to prompt the user to further perform electrocardiographic signal measurement. Moreover, the symbol of ECG measurement may flash until the user starts to perform electrocardiographic signal measurement, so as to better urge the user. In another preferred embodiment, another symbol may be lit to indicate a possible arrhythmia event, so that the user can perform electrocardiographic signal measurement accordingly. For example, RHYTHM in FIG. 33 indicates that there detects a problem related to heart rate, such as AF, tachycardia, bradycardia, and pause. In another preferred embodiment, the symbol of ECG measurement and the symbol of RHYTHM are lit simultaneously, as shown in FIG. 34, to prompt the user to perform electrocardiographic signal measurement. The present invention places no limitation thereto and allows various possibilities, as long as the user is clearly informed of the possible arrhythmia event and prompted to perform electrocardiographic signal measurement.

Herein, the notification may be presented as an acoustic signal, a visual signal, and/or a tactile signal, without limitation. For example, the information may be displayed in a screen, as described previously, by variation of symbols or text. Additionally, the information may be presented to the user in other ways, such as light change, voice or sound, or vibration, without limitation, as long as the user can clearly understand the information. Alternatively, the notification may be presented through an external apparatus. For example, it may be wirelessly transmitted to a smart phone, a tablet computer, or a smart watch to display, so as to allow the user to get notification even more convenient.

After generation of the notification, the disclosed apparatus for monitoring cardiovascular health enters into a state where it is capable of measuring electrocardiographic signals, so as to allow a user to perform electrocardiographic signal measurement by contact the electrodes. Herein, the operation process may vary depending on the locations of the electrodes. For example, when there is an electrode combined on the cuff, the user only needs to further contact the other electrode by, for example, wearing the ear-worn structure, the finger-worn structure, the wrist-worn structure, pressing the electrode on the housing surface, or pressing the electrode at the outer side of the band. Alternatively, when there is no electrode on the cuff, electrocardiographic signal measurement may be performed using two additional electrodes. For example, the user may do this by wearing both an ear-worn structure and a finger-worn structure, wearing two finger-worn structures on two hands, wearing an ear-worn structure and using his/her finger to press an electrode on the housing surface, or wearing a finger-worn structure at his/her one hand and using the other hand to press an electrode on the housing surface. Alternatively, when the two electrodes are both located on the housing surface, the user may directly hold the housing with one hand contacting one of the electrodes, and make the other electrode contact the other hand or the torso, so as to perform electrocardiographic signal measurement. Alternatively, when the two electrodes are both located on another holdable housing connected to the housing, the user may hold the holdable housing with one hand contacting one of the electrodes, and make the other electrode contact the other hand or the torso to perform electrocardiographic signal measurement. The design and configuration of the electrodes may vary according to real demands, without limitation.

Additionally, electrocardiographic signal measurement may be started in different ways. For example, the user may decide by himself/herself when to start measurement and press the start button. Alternatively, the measurement starts automatically when the contact between the electrodes and the skin is checked through impedance check and confirmed as ready for measurement. For example, once the apparatus enters a state capable of measuring electrocardiographic signals, impedance check is performed. When the user properly wears and/or contacts the electrodes, and the result of impedance check confirms electrode contact is ready for electrocardiographic signal measurement, the measurement starts automatically, for example, the user is notified that electrode contact has been established and electrocardiographic signal measurement is about to start by screen display or sound notification. Alternatively, after the apparatus enters the state capable of measuring electrocardiographic signals, as described previously, a sensor first check whether the electrodes are properly located, and then impedance check is performed. Afterward, if the result of impedance check confirms the contact between the electrodes and the skin is ready, the measurement starts automatically. The present invention places no limitation thereto, and there are various options.

After the electrocardiogram is obtained, the electrocardiogram analyzing unit analyzes the obtained electrocardiogram, so as to provide more information of heart conditions. Since the electrocardiogram provides detailed electrical activities of heart, by analyzing the electrocardiogram, it is possible to first verify whether the possible arrhythmia event suggested by the arrhythmia detecting unit is true, and to subsequently identify the type of arrhythmia, for example, to distinguish PAC and PVC, and to accurately determine symptoms such as bradycardia, tachycardia, AF, and pause. It is also possible to know whether there are other heart disorders. For example, the ST value indicates whether there is a symptom of myocardial infarction, and the amplitude of the QRS wave indicates whether there is ventricle hypertrophy. In this way, the user is aware of his/her heart's conditions according to the electrocardiogram as soon as a possible arrhythmia event is detected, and can use this information as a reference for medical consultation.

In the aforesaid, the present invention provides an apparatus for monitoring cardiovascular health which provides two functions, namely blood pressure measurement and electrocardiographic signal measurement. It also incorporates the installation step of electrodes required by electrocardiographic signal measurement into the conventional process for using a sphygmomanometer to measure blood pressure, thereby adding the function of electrocardiographic signal measurement without increasing operation complexity. In addition, in virtue of the prevalence of sphygmomanometers in general households, electrocardiographic signal measurement at home can be increasingly adopted by people by using the present invention. Moreover, based on the relevance between blood pressure values and electrocardiograms, the present invention can provide more cardiovascular information as reference for home healthcare and clinical treatment.

Moreover, the present invention further proposes special designs and locations for the arrangement of electrocardiographic electrodes, so as to improve the obtained electrocardiographic signal quality, thereby facilitating of more accurate analytic results. Wherein by wearing a wearable structure that actively applies force to the skin, such as an ear-worn structure, a finger-worn structure, a wrist-worn structure, and structures establishing skin contact when the cuff is set up, such as a carrying structure on the housing surface, and an electrode structure combined with the cuff, the present invention ensures stable contact between the electrode and the skin, and minimizes interference from electromyographic signals and artifacts.

The present invention also provides a mechanism for pre-screening whether there is a possible arrhythmia event and then verifying the possible arrhythmia event by measuring an electrocardiogram. This mechanism allows a user to be aware of a possible arrhythmia event without changing the operation flow of blood pressure measurement he/she is used to. This mechanism also employs a notification to prompt the user to perform electrocardiographic signal measurement when the possible arrhythmia event is detected, so as to immediately get accurate arrhythmia-related information. In addition, since the electrodes required for electrocardiographic signal measurement are integrated with the sphygmomanometer, the user only needs to contact the electrode directly and the measurement can be performed. Briefly, as compared to the prior-art devices and approaches, the present invention is more convenient to use and requires less cost, thus being of great help to people who care about their cardiovascular health.

1. An apparatus for monitoring cardiovascular health, comprising:
a housing;
a control circuitry, comprising a processor and contained in the housing;
an inflatable cuff, configured to surround a user's one upper limb;
a pump, contained in the housing;
at least a first electrode and a second electrode; and
an ear-worn structure, having the first electrode mounted thereon,
wherein,
when performing a blood pressure measurement, the processor controls the pump to inflate and deflate the cuff, for measuring blood pressures of the user; and
when performing an electrocardiographic signal measurement, through mounting the ear-worn structure on an ear of the user, the first electrode contacts the skin of the ear or around the ear, and through mounting the cuff surrounding the upper limb, the second electrode contacts the skin of the upper limb, thereby enabling the processor to acquire electrocardiographic signals through the first electrode and the second electrode.

2. The apparatus of claim 1, wherein the ear-worn structure is implemented as one of a group consisting of: an ear clamp, an ear plug, and an ear hook.

3. The apparatus of claim 1, wherein the second electrode is located on an inner surface of the cuff to contact the skin of the surrounded upper limb; or the second electrode is combined with an edge of the cuff to contact the skin of the surrounded upper limb.

4. The apparatus of claim 1, wherein the second electrode is mounted on a surface of the housing, and the housing is carried by the cuff, and the second electrode is implemented to position on a carrying structure and the carrying structure is located on the housing, so that the second electrode contacts the skin of the upper limb when the cuff encompasses the upper limb; or wherein the second electrode is implemented to position on a carrying structure and the carrying structure is located on another housing combined with the housing, so that the second electrode contacts the skin of the upper limb when the cuff encompasses the upper limb.

5-6. (canceled)

7. The apparatus of claim 4, wherein the another housing and the housing are implemented to mechanically combine with and electrically connect to each other through a pair of connectors, and the first electrode is connected to the another housing through a connecting wire.

8. (canceled)

9. The apparatus of claim 1, further comprising a communication module for performing wired or wireless communication with an external apparatus, and wherein the external apparatus provides one or more of functions including: control, display, storage, and analysis.

10. The apparatus of claim 1, wherein the processor further performs an HRV analysis on the electrocardiographic signals, so as to generate information of autonomic nervous system activity of the user.

11. An apparatus for monitoring cardiovascular health, comprising:
a housing;
a control circuitry, comprising a processor, and contained in the housing;
an inflatable cuff, configured to surround an upper limb of the user;
a pump, contained in the housing;
at least a first electrode and a second electrode;
a finger-worn structure, having the first electrode mounted thereon,
wherein,
when performing a blood pressure measurement, the processor controls the pump to inflate and deflate the cuff, for measuring blood pressures of the user; and
when performing an electrocardiographic signal measurement, through mounting the finger-worn structure on a finger of the user, the first electrode contacts the skin of the finger, and the second electrode contacts a skin portion of the user other than the upper limb of the finger, thereby enabling the processor to acquire electrocardiographic signals through the first electrode and the second electrode.

12. The apparatus of claim 11, wherein the finger-worn structure is implemented to be one of a group consisting of: a ring, a finger clamp, and a band surrounding a finger.

13. The apparatus of claim 11, wherein the second electrode is located on an inner surface of the cuff to contact the skin of the surrounded upper limb; or the second electrode is combined with an edge of the cuff to contact the skin of the surrounded upper limb.

14. The apparatus of claim 11, wherein the second electrode is located on a surface of the housing, and the housing is carried by the cuff, and the second electrode is implemented to position on a carrying structure and the carrying structure is located on the housing, so that the second electrode contacts the skin of the upper limb when the cuff encompasses the upper limb; or wherein the second electrode is implemented to position on a carrying structure and the carrying structure is located on another housing combined with the housing, so that the second electrode contacts the skin of the upper limb when the cuff encompasses the upper limb.

15-16. (canceled)

17. The apparatus of claim 14, wherein the another housing and the housing are implemented to mechanically combine with and electrically connect to each other through a pair of connectors, and the first electrode is connected to the another housing through a connecting wire.

18. (canceled)

19. The apparatus of claim 11, further comprising a communication module for performing wired or wireless communication with an external apparatus, wherein the external apparatus provides one or more of functions including: control, display, storage, and analysis.

20. The apparatus of claim 11, wherein the processor further performs an HRV analysis on the electrocardiographic signals, so as to generate information of autonomic nervous system activity of the user.

21-29. (canceled)

30. An apparatus for monitoring cardiovascular health, comprising:
a housing;
a control circuitry, comprising a processor, and at least partially contained in the housing;
an inflatable cuff, for carrying the housing and surrounding an upper limb of a user;
a pump, contained in the housing;
a carrying structure, mounted on another housing, which is connected with the housing, wherein the another housing has a indentation;
at least a first electrode and a second electrode, wherein the first electrode is located on the carrying structure, and the second electrode is located inside the indentation;
wherein,
when performing a blood pressure measurement, the processor controls the pump to inflate and deflate the cuff for measuring the user's blood pressure; and
when performing an electrocardiographic signal measurement:
the housing and the another housing are configured to mechanically combine with and electrically connect to each other through a pair of connectors; and
the cuff encompasses the upper limb, so that the first electrode contacts the skin of the upper limb through the carrying structure, and the second electrode is contacted by a finger of the other upper limb of the user that is put into the indentation, thereby enabling the processor to acquire electrocardiographic signals through the first electrode and the second electrode.

31. A method for detecting arrhythmia through measuring arterial pulses and electrocardiographic signals, the method being executed by an apparatus for monitoring cardiovascular health and comprising the following steps:
measuring blood pressures and plural consecutive arterial pulses of a user using a blood pressure monitoring unit and a cuff of the apparatus for monitoring cardiovascular health;
calculating a time interval between each two consecutive arterial pulses, so as to obtain a time-sequence characteristic, and comparing the time-sequence characteristic to at least one of predetermined arrhythmia time-sequence characteristics;
determining a possible arrhythmia event when matched;
generating a notification to inform the user of the possible arrhythmia event and prompt the user to perform electrocardiographic signal measurement;
the apparatus for monitoring cardiovascular health entering into a state capable of measuring electrocardiographic signals;
the user performing electrocardiographic signal measurement using two electrocardiographic electrodes of the apparatus for monitoring cardiovascular health, so as to obtain an electrocardiogram;
storing the electrocardiogram; and
analyzing the electrocardiogram, for providing arrhythmia related information and other information available from the electrocardiogram.

32. The method of claim 31, wherein the predetermined arrhythmia time-sequence characteristics include premature beats, atrial fibrillation, bradycardia, tachycardia, and pause, and the arrhythmia related information includes types of arrhythmia.

33. (canceled)

34. The method of claim 31, wherein the notification is implemented as acoustic signals, visual signals, and/or tactile signals, and wherein the acoustic signal is implemented as sound change and/or voice, and the visual signal is implemented as one or more of a group consisting of: texts, graphs, and light.

35-38. (canceled)

39. An apparatus for monitoring cardiovascular health, for measuring blood pressure and for detecting arrhythmia through measuring arterial pulses and electrocardiographic signals, wherein the apparatus comprising:
a blood pressure monitoring unit;
an inflatable cuff, connected to the blood pressure monitoring unit and configured to encompass a limb of a user, for measuring blood pressures and plural consecutive arterial pulses of the user;
an arrhythmia detecting unit, calculating a time interval between each two consecutive arterial pulses to obtain a time-sequence characteristic, comparing the time-sequence characteristic to at least one of predetermined arrhythmia time-sequence characteristics, and determining a possible arrhythmia event when matched, wherein, after the possible arrhythmia event is determined, the apparatus for monitoring cardiovascular health enters a state capable of measuring electrocardiographic signals;
a notification generating unit, generating a notification to inform the user of the possible arrhythmia event and prompting the user to perform electrocardiographic signal measurement;
an electrocardiographic signal measuring unit, comprising at least two electrodes, for performing electrocardiographic signal measurement using the at least two electrode, so as to obtain an electrocardiogram;
a storage unit, storing the obtained electrocardiogram; and
an analysis unit, analyzing the electrocardiogram so as to provide arrhythmia related information and other information available from the electrocardiogram.

40. The apparatus of claim 39, wherein the predetermined arrhythmia time-sequence characteristics comprises premature beats, atrial fibrillation, bradycardia, tachycardia, and pause, the arrhythmia related information comprises types of arrhythmia, and the notification is implemented as one or more of a group consisting of: acoustic signal, visual signal, and tactile signal.

41-42. (canceled)

43. The apparatus of claim 39, wherein at least one of the at least two electrodes is located on one of structures including: an ear-worn structure, a finger-worn structure, and a wrist-worn structure; or the apparatus further comprises a housing, and at least one of the at least two electrodes is located on a surface of the housing; or at least one of the at least two electrodes is located on the cuff.

44-45. (canceled)
